(19) United States
(12) Patent Application Publication
(10) Pub. No.: US 2024/0232859 A9
(43) Pub. Date: Jul. 11, 2024

(54) Medical Data Non-Fungible Tokens (NFTs)

(71) Applicant: STATE FARM MUTUAL AUTOMOBILE INSURANCE COMPANY, Bloomington, IL (US)

(72) Inventors: Joseph Robert Brannan, Bloomington, IL (US); Brian N. Harvey, Bloomington, IL (US); Edward W. Breitweiser, Bloomington, IL (US); Joseph P. Harr, Bloomington, IL (US)

(21) Appl. No.: 18/187,631

(22) Filed: Mar. 21, 2023

Related U.S. Application Data
(60) Provisional application No. 63/418,884, filed on Oct. 24, 2022.

(51) Int. Cl. G06Q 20/36; G06Q 50/18; G16H 10/60

(52) U.S. Cl. CPC G06Q 20/3672; G06Q 50/188; G16H 10/60

(57) ABSTRACT

The following relates generally to implementing a medical data non-fungible token (NFT). In some embodiments, one or more processors receive medical data of a plurality of users, and store the medical data of the plurality of users in a first storage location in a medical data database. The one or more processors may then detect a request to purchase a data feed for one or more types of medical data, and identify medical data corresponding to the request in the medical data database. The one or more processors may then store the identified medical data in a second storage location, and mint a medical data NFT that indicates the second storage location.

This application claims the benefit of U.S. Provisional Patent Application No. 63/418,884, entitled “Medical Data Non-Fungible Tokens (NFTS),” filed Oct. 24, 2022. U.S. Provisional Patent Application No. 63/418,884 is hereby expressly incorporated by reference herein in its entirety.

### FIELD

The present disclosure generally relates to providing data owners control over their medical data, and, more particularly, to minting medical data non-fungible tokens (NFTs) that control access to data owners' medical data.

### BACKGROUND

Many companies rely on large amounts of medical data to improve existing medical treatments, and/or innovate new medical treatments. However, such large amounts of medical data may be difficult for the companies to acquire. Furthermore, many individuals might be willing to sell their medical data (or specific segments of their medical data) to such companies, but may not have an efficient and secure mechanism to do so.

The systems and methods disclosed herein provide solutions to these problems and may provide solutions to the ineffectiveness, insecurities, difficulties, inefficiencies, encumbrances, and/or other drawbacks of conventional techniques.

### SUMMARY

The present embodiments relate to, inter alia, implementing medical data non-fungible tokens (NFTs). For example, many individuals may not sell their medical data to research companies because the individuals do not have an efficient and secure mechanism to do so and/or because they only wish to sell particular types of medical data (e.g., an individual is willing to sell heartrate data, but not medical history). The following embodiments provides an elegant solution that includes minting medical data NFTs that provide an efficient, secure, and customizable mechanism for selling and/or sharing medical data.

In one aspect, a computer system for implementing a medical data non-fungible token (NFT), may be provided. The computer system may include one or more local or remote processors, sensors, transceivers, servers, memory units, mobile devices, augmented reality glasses or headsets, extended or virtual reality headsets, smart glasses or watches, wearables, and/or other electronic or electrical components. In one instance, the computer system may include: a medical data database configured to store medical data, and/or one or more processors; and/or one or more memories coupled to the one or more processors. The one or more memories may include computer executable instructions stored therein that, when executed by the one or more processors, may cause the one or more processors to: (1) receive medical data of a plurality of users; (2) store the received medical data of the plurality of users in a first storage location, wherein the first storage location is in the medical data database; (3) detect a request from a requestor to purchase a data feed for one or more indicated types of medical data of a user of the plurality of users; (4) identify, in the medical data database, the medical data for the user that matches the one or more indicated types of medical data; (5) store the identified medical data corresponding to the user at a second storage location; and/or (6) mint a medical data NFT that indicates the second storage location, wherein minting the medical data NFT adds the medical data NFT to a digital wallet associated with the requestor. The computer system may include additional, less, or alternate functionality, including that discussed elsewhere herein.

In another aspect, a computer-implemented method for implementing a medical data non-fungible token (NFT) may be provided. The method may be implemented via one or more local or remote processors, sensors, transceivers, servers, memory units, mobile devices, augmented reality glasses or headsets, extended or virtual reality headsets, smart glasses or watches, wearables, and/or other electronic or electrical components. In one instance, the computer-implemented method may include: (1) receiving, via one or more processors, medical data of a plurality of users; (2) storing, via the one or more processors, the received medical data of the plurality of users in a first storage location, wherein the first storage location is in a medical data database; (3) detecting, via the one or more processors, a request from a requestor to purchase a data feed for one or more indicated types of medical data of a user of the plurality of users; (4) identifying, via the one or more processors, in the medical data database, the medical data for the user that matches the one or more indicated types of medical data; (5) storing, via the one or more processors, the identified medical data corresponding to the user at a second storage location; and/or (6) minting, via the one or more processors, a medical data NFT that indicates the second storage location, wherein minting the medical data NFT adds the medical data NFT to a digital wallet associated with the requestor. The method may include additional, fewer, or alternate actions, including those discussed elsewhere herein.

### BRIEF DESCRIPTION OF THE DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The figures described below depict various aspects of the applications, methods, and systems disclosed herein. It should be understood that each figure depicts an embodiment of a particular aspect of the disclosed applications, systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 depicts an exemplary computer system for utilizing medical data NFTs to control access to user medical data, according to one embodiment.

FIG. 2 depicts an exemplary user interface presented to a data owner.

FIG. 3A depicts an exemplary user interface presented to a purchaser of medical data, including purchasing medical data via data owner smart contracts.

FIG. 3B depicts an exemplary user interface presented to a purchaser of medical data, including purchasing medical data via sending offers to users.

FIG. 4 illustrates exemplary nodes, and an exemplary distributed ledger.

FIG. 5 illustrates exemplary nodes, and an exemplary transaction flow on a distributed ledger network.

FIG. 6 illustrates exemplary components of a node on a distributed ledger network.

FIG. 7 shows an exemplary computer-implemented method or implementation of minting a medical data NFT.

### DETAILED DESCRIPTION

Broadly speaking, the following provides an efficient mechanism for data owners (e.g., “users”) to control which types of medical data are available for sale and how the available medical data is able to be utilized (e.g., for research purposes, etc.). In some embodiments, the system is customizable such that the users may specify what types of medical data they would like to sell, as well as specify prices and/or other terms of use associated with their medical data. Likewise, in some embodiments, prospective purchasers of the medical data (e.g., medical research companies, etc.) may purchase certain types of medical data, as well as buy medical data at prices that they specify. To accomplish this, some embodiments mint a medical data NFT.

Broadly speaking, an NFT is a record on a distributed ledger that includes a unique token that identifies the record thereby making the record “non-fungible.” Typically, NFTs are associated with a digital asset (e.g., an image, an audio file, a digital ticket, and/or other types of digital assets). In the instant techniques, the digital asset may be medical data and/or or stream of medical data provided by a data owner. For example, a data owner may offer for sale a data feed of heart rate data. In this example, a NFT may be minted to create a non-fungible right to view the data owner's heart rate data.

The NFT may include an identifier associated with an owner of the NFT (e.g., an identifier of a digital wallet on the distributed ledger that holds the NFT, an identifier of the entity that owns the NFT, etc.) and/or an indication of the medical data associated with the NFT. Additionally, the NFT may also include an indication a set of information describing the digital asset. As one example, the NFT may indicate a location (e.g., an HTTP address, an FTP address, an IP address, a IPFS address, and/or other types of addresses) at which the digital asset may be accessed. As another example, the NFT may include an indication of the data stored at the location (e.g., biographical data, data from a particular type of sensor, and so on).

According to certain aspects, the NFT may also include a set of rights and/or restrictions associated with the data located at the indication location. For instance, the NFT may specify that the owner of the NFT has rights to use, copy, and/or transfer the digital asset. In some embodiments, the rights are further narrowed to particular classes. For example, the usage rights may be divided into usage may include research purposes, commercial purposes, treatment purposes, and so on. As another example, the transfer rights may limit the types of entities (such as medical institutions, advertisers, medical device manufacturers, etc.) to which the NFT may be transferred and/or re-sold.

In some embodiments, the NFT is associated with a smart contract that enforces the set of rights and/or restrictions associated with the NFT. Accordingly, when a participant in the distributed ledger submits a request to perform an interaction with the NFT (e.g., to access the digital asset, to transfer the NFT, etc.), the smart contract may validate that the requested interaction is permitted prior to executing the request.

In one example, in response to a request to access the medical data, the smart contract ensures that request is from the NFT owner. For example, the smart contract may validate that a digital signature applied to the request authenticates the requestor as the NFT owner. As another example, a NFT owner may submit a NFT transfer request. In this example, the smart contract may validate that the transfer complies with the set of restrictions prior to executing the transfer.

According to certain additional aspects, the NFT may also specify obligations associated with NFT ownership. For example, the data owner may request weekly, monthly, and/or annual payment for making their medical data available. Accordingly, the smart contract may enforce the payment terms associated with the NFT. For example, the smart contract may transfer a specified amount of digital currency from at a digital wallet associated with the NFT to the digital wallet associated with the data owner. In this example, the data owner digital wallet identifier may be included in the NFT and/or configured into the smart contract associated therewith. In some embodiments, if the NFT submits a transfer request mid-payment period, the smart contract may execute to perform a pro-rated transfer of currency to the data owner prior from the digital wallet of the transferor prior to completing the transfer.

Additionally, unlike conventional NFTs, the data owner may reserve the right to withdraw the NFTs from the marketplace, even if data owner does not own the corresponding NFT. For example, the smart contract may be configured to detect revocation requests written to the distributed ledger. Upon validating that the revocation request was submitted by the data owner, the smart contract may transfer ownership of the NFT to the data owner, update the access rights of the NFT smart contract to prevent third party access to the digital asset, and/or cause the medical data to be deleted from the location indicated in the NFT. As a result, the instant techniques may relate to an unconventional marketplace for NFTs where data owners are able to control access to their data even after transferring the NFT to a third party.

### Exemplary Computer System

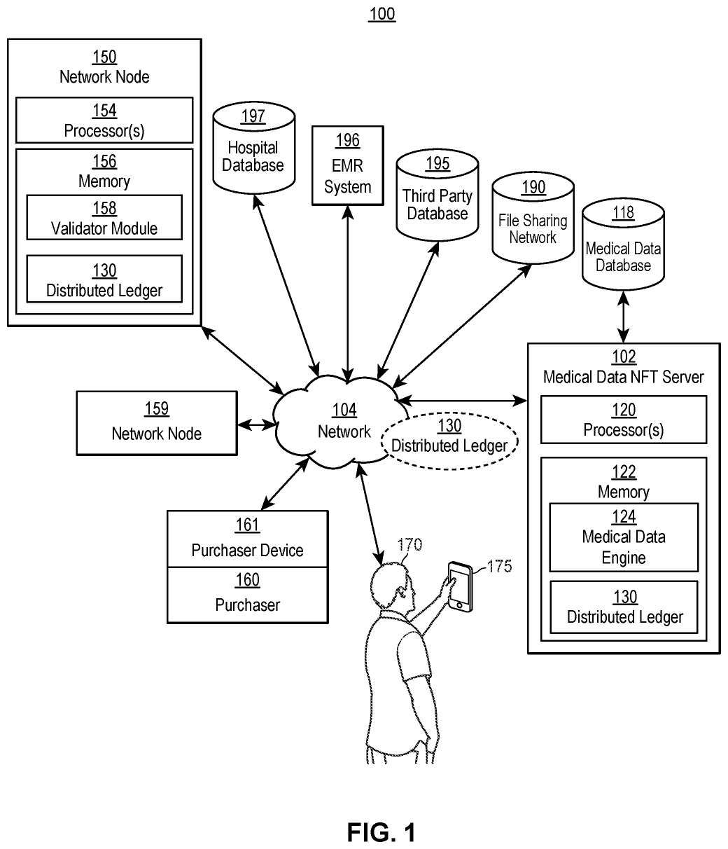

FIG. 1 shows an exemplary computer system 100 for utilizing medical data NFTs to control access to user medical data in which the exemplary computer-implemented methods described herein may be implemented. The high-level architecture includes both hardware and software applications, as well as various data communications channels for communicating data between the various hardware and software components. Although the example system 100 illustrates only one of each of the components, any number of the example components are contemplated (e.g., any number of users, user devices, medical data NFT servers, nodes, medical research companies, hospital databases, EMR systems, third party databases, file sharing networks, etc.). The illustrated example components may be configured to communicate, e.g., via a network 104 (which may be a wired or wireless network, such as the internet), with any other component.

By way of brief overview, the distributed ledger 130 may maintain records of NFTs and/or smart contracts associated therewith that facilitate the user-controlled sale and/or sharing of medical data.

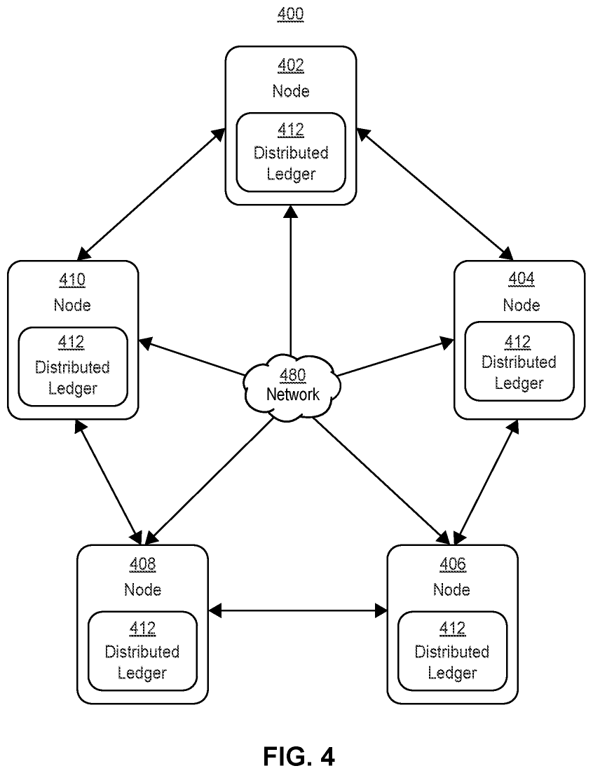

The distributed ledger 130, as will be described in further detail elsewhere herein (e.g., with respect to FIGS. 4-6), may be maintained by a plurality of nodes, such as nodes 150, 159. According to embodiments, the nodes 150, 159 may be a combination of hardware and software components, also as described in more detail below with reference to FIGS. 4-6 (e.g., the nodes 150, 159 correspond to the nodes of FIGS. 4-6). The nodes 150, 159 may each include a memory 156, one or more processors 154, such as a microcontroller or a microprocessor, and other components not shown in FIG. 1 (e.g., a random-access memory (RAM), and/or an input/output (I/O) circuit), all of which may be interconnected via an address/data bus.

The memory 156 and/or RAM may store various applications for execution by the one or more processors 154. For example, a user interface application may provide a user interface to the node 150, which user interface may, for example, allow the system administrator to configure, troubleshoot, and/or test various aspects of the node's operation.

The memory 156 may be tangible, non-transitory memory and may include any types of suitable memory modules, including RAM, read-only memory (ROM), flash memory, other types of persistent memory, etc. The memory 156 may store, for example, instructions executable on the processors 154 for a validator module 158.

The validator module 158 may validate changes to the blockchain (e.g., when a new transaction and/or block is created, the state of a smart contract, etc.) according to a set of consensus rules. The consensus rules may depend on the information being tracked by the blockchain and may include rules regarding the chain itself. For example, a consensus rule may include that the originator of a change supply a proof-of-identity such that only approved entities may originate changes to the chain. Consensus rules may include a mechanism to determine the order in which new blocks are added to the chain (e.g., through a proof-of-work system, proof-of-stake, etc.).

The validator module 158 may update distributed ledger data to the distributed ledger 130 if upon satisfaction of the consensus rules. For example, the validator module 158 may generate a new block of validated transactions to include in the distributed ledger 130 and/or broadcast a block of transactions to other nodes. If the update does not satisfy the consensus rules, the validator module 158 may disregard the update such that the update is not propagated to other nodes.

The distributed ledger 130 may be accessed by user 170 through the user device 175. The user device 175 may be any kind of user device. Examples of user devices include computers, tablets, phablets, smart phones, smart watches (and/or other types of heart rate monitors), medical monitors, cameras (e.g., smart cameras in a smart home, etc.), a virtual reality (VR) headset, an augmented reality (AR) headset, etc. The user device 170 may execute one or more applications that interface with the distributed ledger 130. For example, the applications may enable the user to define access controls associated with the medical data, assign pricing for the medical data, approve/reject offers for their medical data, add additional medical data feeds to their user profile, and so on. In some embodiments, the user device 175 is itself a node of the distributed ledger 130.

It should be appreciated that while FIG. 1 depicts a single distributed ledger 130, in some embodiments, multiple distributed ledgers are implemented. For example, in some embodiments, the distributed ledger 130 may be configured to process purchase requests and/or payment for medical data NFTs and include a sidechain for the transfer of the medical data NFTs and/or the secure access to the data associated with the medical data NFTs.

The medical data NFT server 102 may include one or more processors 120 such as one or more microprocessors, controllers, and/or any other suitable type of processor. The medical data NFT 102 may further include a memory 122 (e.g., volatile memory, non-volatile memory) accessible by the one or more processors 120, (e.g., via a memory controller). The one or more processors 120 may interact with the memory 122 to obtain and execute, for example, computer-readable instructions stored in the memory 122. Additionally or alternatively, computer-readable instructions may be stored on one or more removable media (e.g., a compact disc, a digital versatile disc, removable flash memory, etc.) that may be coupled to the medical data NFT 102 to provide access to the computer-readable instructions stored thereon. In particular, the computer-readable instructions stored on the memory 122 may include instructions for executing various applications, such as medical data engine 124.

In operation, the medical data engine 124 may facilitate the secure sale and/or sharing of medical data. For example, the medical data engine 124 may receive medical data from users, such as the user 170, and maintain the medical data in the medical data database 118 (e.g., in a first storage location located in the medical data database 118). The medical data stored in the medical data database 118 may come from any suitable source. For example, the medical data may come from the user device 175, the electronic medical record (EMR) system 196, the hospital database 197, etc. In some examples, the user device 175 gathers the medical data (e.g., a smart watch gathers heart rate data of the user 170, a medical monitor gathers blood oxygen levels, etc.). Additionally or alternatively, the medical data may come from a database, such as the EMR system 196, hospital database 197, or any other kind of database (not shown in the example of FIG. 1), such as a primary care provider (PCP) database, etc.

In some embodiments, an application executing on the user device 175 may be configured to interface with the medical data engine 124 to securely sell access to their medical data. To this end, the medical data engine 124 may be configured to generate a user interface for presentation by the application executing on the user device 175.

### Exemplary User Interfaces

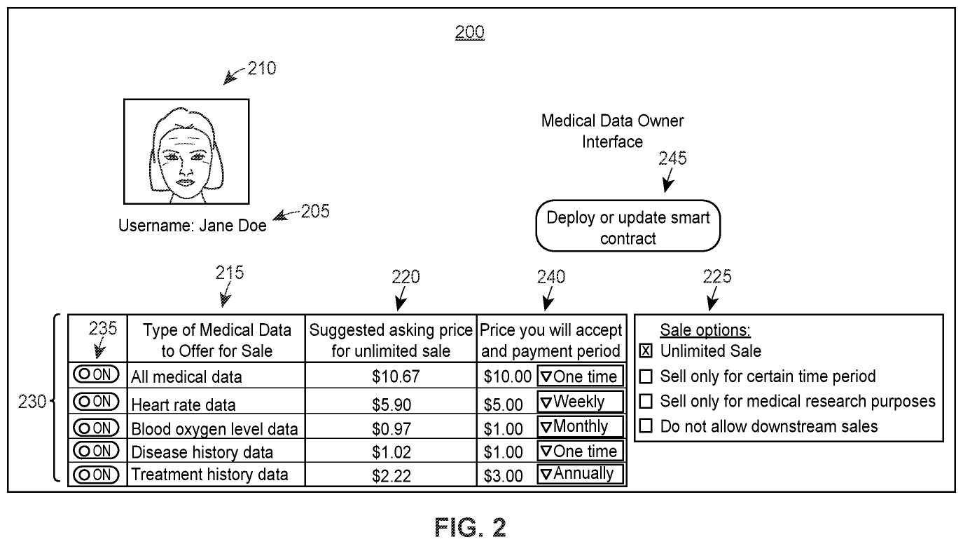

With simultaneous reference to FIG. 2, illustrated is an exemplary user interface 200 presented via the user device 175 via which the user 170 is able to deploy and/or update a data owner smart contract for the secure sale of user-specified types of medical data. As illustrated, the exemplary user interface 200 includes a username 205 and a profile picture 210.

The exemplary user interface 200 also includes a selection interface 230 that enables the user to select and/or update the types of medical data to sell. For example, the selection interface 230 may be populated by the medical data engine 124 based upon the available types of medical data associated with the user 170 as maintained at the medical data database 118. That is, as the user 170 updates their user profile to include the additional and/or fewer sources of medical data, the selection interface 230 may automatically adjust the presented types of medical data in accordance therewith.

For the presented types of medical data, the selection interface 230 may include an indication 215 of the type of medical data to offer for sale, and/or an indication 220 of a suggested asking price. According to certain aspects, the medical data engine 124 generates the indication 220 based upon distributed leger data (such as offer prices across a plurality of data owner smart contracts that list that type of medical data and/or a NFT transaction history for NFTs that include that type of medical data). Accordingly, the medical data engine 124 may dynamically calculate the suggested values upon receiving a request from the user device 170 to present the user interface 200.

Additionally, for each listed type of medical data, the selection interface 230 may also include a toggle element 235 that enable the user 170 to control whether or not the type of medical data is available for sale. If the type of medical data is available for sale, the user interface 200 may include a customization interface 225 via which the user is able to place, use and/or transfer restrictions on NFTs that include the type of medical data. For example, in the illustrated example, the customization interface 225 may enable an unlimited sale (e.g., selling the medical for any period of time to any party, etc.), sale for only a certain time period (e.g., a research company will only have access to the data for a certain time period), sale only for medical research purposes (e.g., not selling for marketing purposes, etc.), and a sale that prevents downstream sales (e.g., company that purchases the data is not allowed to resell it, etc.). It should be appreciated that the medical data engine 124 may dynamically adjust the indication 220 based upon the customizations selected via the customization interface 225. That is, the medical data engine 124 may filter the distributed ledger data such that the indication 220 may be further derived based upon sales and/or offers for sale with the same or similar customization options.

Additionally, the selection interface 230 may include a user price field 240 via which the user 170 sets the price and/or payment period (e.g., one-time, weekly, monthly, annually, etc.) for the type of medical data. In the illustrated example, the user 170 sets the price by typing the price into the field, and sets the payment period via a dropdown menu. In some embodiments, the selection interface 230 may include multiple copies of the same type of medical data. For example, the user 170 may set a first price for restricted access to the type of medical data, and a second, higher, price for unlimited access to the type of medical data. Accordingly, the selection interface 230 may enable the user 170 to set different prices and/or payment periods for different packages of usage rights for a particular type of medical data.

The user interface 200 also includes an element 245 that deploys and/or updates a smart contract on the distributed ledger 130 in accordance with the indications provided via the selection interface 230 and/or the customization interface 225. After deployment, the smart contract may self-execute to respond to purchase requests written to the distributed ledger 130. More particularly, the smart contract may detect a purchase request that agrees to the payment and/or usage restriction terms for a type of medical data and mint an NFT that provides access to the purchased data. Because the smart contract is configured and deployed by the user 170, only medical data NFTs that are specifically authorized by the user 170 may be minted and transferred via the distributed ledger 130.

The medical data engine 124 may also present medical data buying options to a purchaser 160 or prospective purchaser of the medical data, such as a medical research company, and such as via the purchaser device 161. The purchaser device 161 may be any suitable device, such as a computer, a server, a tablet, a phablet, a smart phone, etc.

In some embodiments, the purchaser may view multiple options for purchasing the medical data. As a first option, the purchaser may purchase medical data according to the terms included the data owner smart contracts. For example, the purchaser may purchase the data according to the prices specified by the user via the interface 200. As a second option, the purchaser may send offers to the user (assuming the user affirmatively opts in to receive such offers). The offers may include any offer terms, such as prices, types and/or subtypes of medical data, downstream sale rights, time period purchased for, etc.

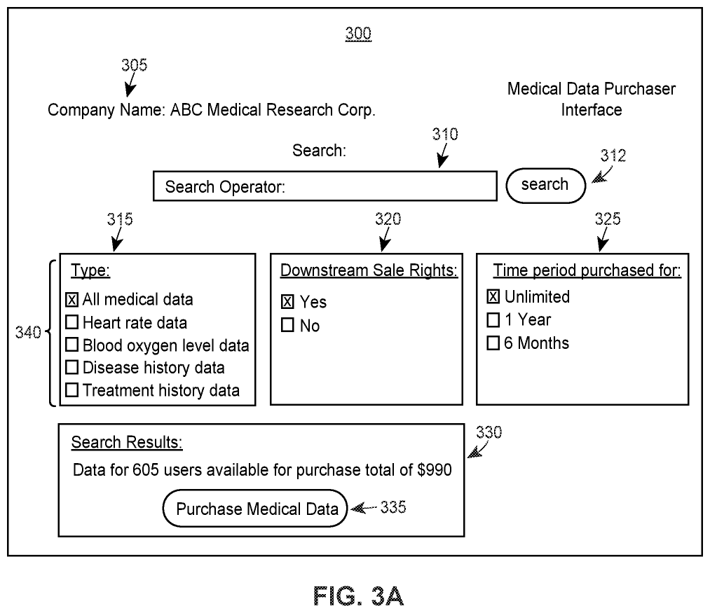

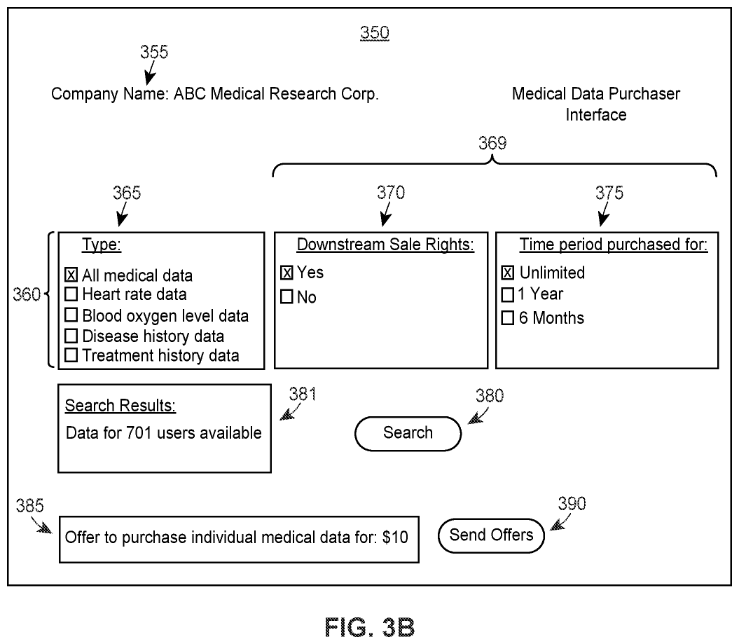

To this end, FIG. 3A depicts an exemplary user interface 300 presented via the purchaser device 161 for purchasing medical data via data owner smart contracts. In the illustrated example, the user interface 300 displays a company name 305, and presents search options.

The exemplary user interface 300 may be configured to conduct a search of medical data available for purchase via deployed data owner smart contracts (such as those deployed via the user interface 200 of FIG. 2). In some embodiments, the user interface 300 may include a search bar 310 for entering a search operator (such as a text description of a type of medical data).

Additionally, the user interface 300 may include a selection interface 340 that enables the purchaser to implement one or more types of search filters. For example, the selection interface 340 may be populated by the medical data engine 124 based upon the available types of medical data and/or usage restrictions thereon generally supported by the medical data engine 124. In some embodiments, the medical data engine 124 may require that available types of medical data and/or usage restrictions thereon are included in a predetermined number of data owner smart contracts (e.g., at least 100 data owner smart contracts) before its inclusion in the selection interface 340.

More particularly, as illustrated, the selection interface 340 may include a medical data type filter interface 315, a downstream rights filter interface 320, and a time period filter interface 325. It should be appreciated that the selection interface 340 may include other filter interfaces based upon the types of data and/or usage restrictions supported by the medical data engine 124 (such as purchase price, demography data, etc.). The user interface 300 may also include a search button 312 that initiates a search based upon the search operators included in the search bar 310 and/or the filters selected via the selection interface 340.

In response to the search button 312 being activated, the medical data engine 124 may conduct the indicated search. Accordingly, the medical data engine 124 may search the data owner smart contracts on the distributed ledger 130 to identify data owner smart contracts that offer medical data for sale in a manner that complies with the search criteria provided via the user interface 300.

After conducting the search, the medical data engine 124 may be configured to present an indication of the search results via the user interface 300. In the illustrated user interface 300, the medical data engine 124 presents an indication 330 of the aggregate search results. For example, the indication 330 may include a number of users with medical data that may be purchased in a manner that complies with the search criteria. Advantageously, this may enable bulk purchasers of medical data feeds to purchase multiple NFTs in a streamlined manner.

In alternate embodiments, the user interface 300 may instead present a list of users and/or medical data feeds available therefrom that comply with the search criteria. In these embodiments, the list may be sorted to prioritize the cheapest options. Accordingly, the purchaser may select the most cost-efficient options for obtaining the desired medical data.

Regardless of how the medical data feeds are selected, the user interface 300 may enable the purchase of the selected medical data feeds via a button 335. In response to the purchase button 335 being selected, the medical data engine 124 may initiate the purchase process for the selected medical data feeds. Accordingly, the medical data engine 124 may write a purchase request transaction to the distributed ledger indicating that the purchaser 160 is requesting the purchase of the selected medical data feeds. The nodes of the distributed ledger 130 that enforce the data owner smart contracts may then detect the purchase request transaction and validate that the request is valid and complies with the access restrictions indicated in the data owner smart contracts.

If the request is valid, the data owner smart contracts may then cause a medical data NFT for the requested data feed to be minted, at least in part, by the medical data engine 124. As part of the minting process the medical data engine 124 may store the purchased medical data at a second storage location (e.g., at any of the medical data database 118, a third party database 195, and/or a file sharing network 190). Additionally or alternatively, the medical data engine 124 may deploy a task that is executed by the processors 120 to write the purchased user data to the second storage location as it obtained by the medical data NFT server 102. Accordingly, the medical data engine 124 may configure the medical data NFT to include an indication of the second storage location. The minted medical data NFT may then be added to a digital wallet associated with the purchaser 160.

While exemplary user interface 300 may enable the purchaser 160 to purchase data feeds associated with deployed data owner mart contracts, in some scenarios, the purchaser 160 may want to purchase data feeds not currently available via a data owner smart contract. Accordingly, FIG. 3B depicts an exemplary user interface 350 presented via the purchaser device 161 for sending purchase offers to users. In the illustrated example, the user interface 350 may display a company name 355 and present search options.

According to certain aspects, the search options available via the user interface 350 may be more limited than those available via the user interface 300. To this end, by deploying a data owner smart contract, the user 170 affirmatively indicated the availability of the particular data feeds and/or the rights associated therewith. In the absence of a data owner smart contract, the user 170 may nonetheless configure a user profile to maintain one or more data feeds with the medical data NFT server 102. Accordingly, the user 170 may nonetheless affirmatively indicate a willingness to receive offers for purchase of particular types of medical data feeds associated with their user profile. Thus, the search provided via the user interface 350 may relate to a search of particular user data feeds that users affirmatively indicated are available for purchase.

The exemplary user interface 350 may include a selection interface 360 that enables the purchaser 160 to provide one or more search filters to identify data feeds available for purchase. For example, the filter criteria may be based upon demographic information and/or the available types of medical data as generally maintained at the medical data database 118.

The user interface 350 may enable the purchaser 160 to conduct a search in accordance with the search criteria provided via the selection interface 360 via search button 380. In response, the medical data engine 124 may search the user profiles associated with the medical data NFT server 102 to identify users that have user profiles that include a data feed that matches the search criteria. The user interface 350 may then present the search results, for example, via a text box 381 that specifies a number of users with medical data available according to the specified search criteria. The user interface 350 may then enable the purchaser 160 to select one or more users that are to receive the purchase offer.

Additionally, the exemplary user interface 350 may include an offer customization interface 369 that enables the purchaser to customize the terms associated with their offer. For example, the customization interface 369 may enable the purchaser 160 to customize options 370 for downstream sale rights (e.g., rights allowing the purchaser to resell the medical data, etc.) and/or options 375 for a time period for access to the data. Additionally, the user interface 350 may include a text entry box 385 via which the purchaser 160 enters a price for the indicated medical data feed.

The user interface 350 may also include a button 390 configured to send purchase offers to the selected users and/or in accordance with the customization options. In response, the medical data engine 124 may notify the user of the purchase offer. For example, the medical data engine 124 may generate a push notification via the application executing on the user device 175. The application may then enable the user 170 to accept, reject, and/or propose a counter-offer for their medical data.

At some point, the purchaser 160 and the data owner 170 may agree upon terms for purchase of one or more feeds medical data. Accordingly, the medical data engine 124 may receive an indication of the agreed-upon terms and mint a medical data NFT in accordance therewith. The minted medical data NFT may then be added to a digital wallet associated with the purchaser 160.

### Exemplary Distributed Ledgers for Implementing a Medical Data NFT

FIG. 4 depicts an exemplary distributed ledger system 400 for implementing a medical data NFT. The system 400 may include a distributed ledger 412 (e.g., having one or more distributed ledger layers) and a plurality of nodes 402, 404, 406, 408, and 410 (e.g., each similar to node 150, 159 of FIG. 1). Each node maintains a copy of the distributed ledger 412. As changes are made to the distributed ledger 412, each node receives the change via the network 480 and updates its respective copy of the distributed ledger 412. A consensus mechanism may be used by the nodes 402-410 in the distributed ledger system 400 to decide whether it is appropriate to make received changes to the distributed ledger 412 or to a particular layer of the distributed ledger 412.

Each node in the system therefore has its own copy of the distributed ledger 412, which is identical to every other copy of the distributed ledger 412 stored by the other nodes. The distributed ledger system 400 may be more robust than a central authority database system because of the distributed ledger's decentralized nature. As such, there is no single point of failure on the distributed ledger system 400 as there would be in a centralized system.

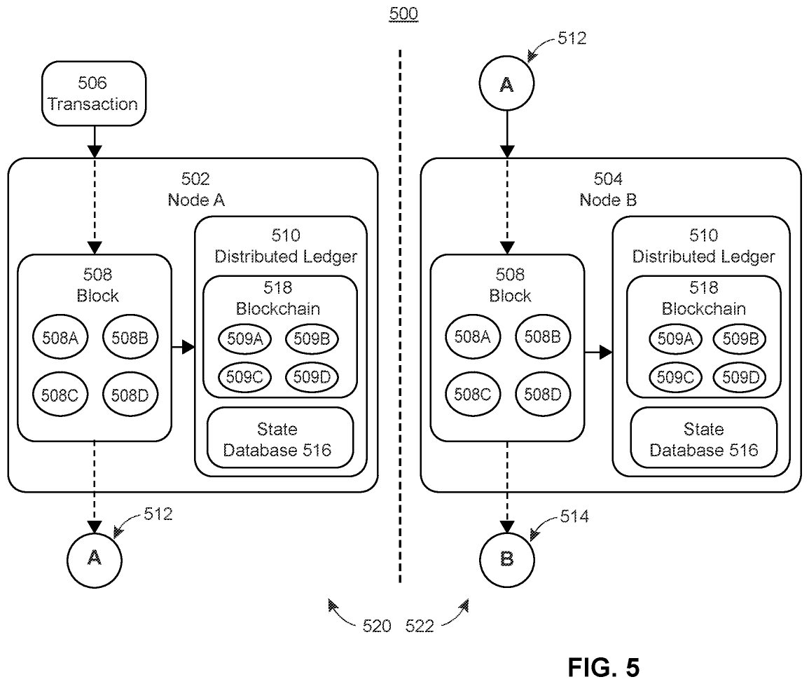

FIG. 5 depicts exemplary nodes and an exemplary transaction flow 500 on a distributed ledger network for implementing a medical data NFT. FIG. 5 includes two time frames 520 and 322 represented by the left and right sides of the dotted line, respectively, Node A 502 (e.g., node 150, etc.) and Node B 504 (e.g., node 159, etc.), a set of transactions 508A-508D, a set of blocks of transactions 509A-509D, a distributed ledger 510, and a blockchain 518.

The block propagation flow 500 may begin with Node A 502 receiving transaction 506 at time 520. When Node A 502 confirms that transaction 506 is valid, Node A 502 may add the transaction to a newly generated block 508. As part of adding the transaction 506 to block 508, Node A 502 may solve a cryptographic puzzle and include the solution in the newly generated block 508 as proof of the work done to generate the block 508. Alternatively, a proof of stake algorithm may be used to generate the block 508, whereby Node A 502 “stakes” an amount of a digital token used on the network, however, the network itself determines the node that will mint the new block. In another implementation, a proof of authority (PoA) algorithm may be used to generate the block 508, where transactions and blocks are validated by approved accounts, known as validators which run software allowing them to record transactions in the distributed ledger.

In other embodiments, the transaction 506 may be added to a pool of transactions until a sufficient number of transactions in the pool exist to form a block or distributed ledger entry. Node A 502 may transmit the newly created distributed ledger entry 508 to the network at time 512. Before or after propagating the distributed ledger entry 508, Node A 502 may add the distributed ledger entry 508 to its copy of the blockchain 518.

While proof of work, proof of stake, and proof of authority are described herein as consensus algorithms for selecting a node to mint a new block, these are merely a few example consensus algorithms and are not intended to be limiting. Additional consensus algorithms may be utilized, such as delegated proof of stake where nodes elect a subset of nodes referred to as delegates to perform validation, and the delegates take turns minting new blocks. Consensus algorithms may also include proof of weight, Byzantine fault tolerance, tangle consensus algorithms, block lattice consensus algorithms, etc. Additionally, quorum slices may be selected where a quorum is a set of nodes that participate in the consensus protocol and a quorum slice is its subset that helps a node in its agreement process. Individual trust decisions may be made by participants in the distributed ledger network to construct a quorum slice. Still further, security circles may be identified which are closed groups of network participants who together can form a quorum to reach a consensus on a transaction and to make further trust decisions.

In any event, the transactions 509A-509D may include updates to a state database 516. The state database 516 may contain current values of variables created by smart contracts deployed on the blockchain 518. Validated distributed ledger entries, such as distributed ledger entry 508, may include transactions effecting state variables in state database 516. At time 522, Node B 504 may receive the newly created distributed ledger entry 508 via the network at 512. Node B 504 may verify that the distributed ledger entry 508 is valid by checking the solution to the cryptographic puzzle provided in the distributed ledger entry 508. If the solution is accurate, then Node B 504 may add the distributed ledger entry 508 to its blockchain 518 and make any updates to the state database 516 as rejected by the transactions in distributed ledger entry 508. Node B 504 may then transmit the distributed ledger entry 508 to the rest of the network at time 514.

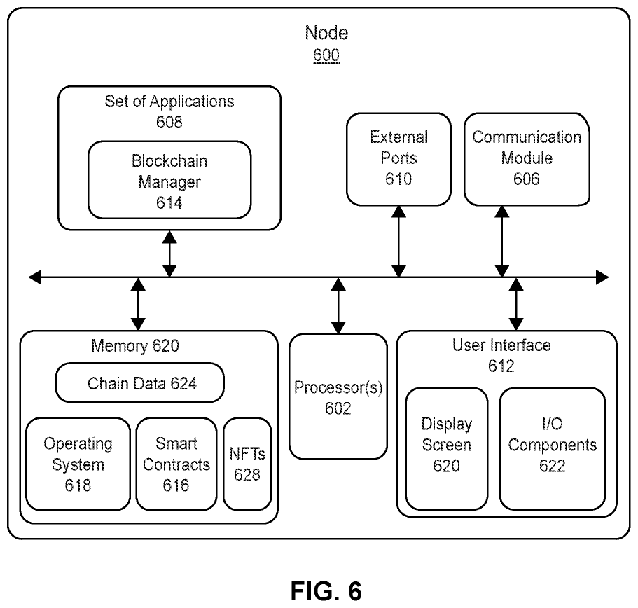

FIG. 6 depicts exemplary components of a node 600 on a distributed ledger network for implementing a medical data NFT. The node 600 may be similar to the nodes 150, 159 described above with reference to FIG. 1. Node 600 may include at least one processor 602, memory 604, a communication module 606 such as a transceiver, a set of applications 608, external ports 610, a blockchain manager 614, smart contracts 616, NFTs 628, an operating system 618, user interface 612, display screen 620, and/or I/O components 622. In some embodiments, the node 600 may generate a new block of transactions, or may broadcast transactions to other nodes via the communication module 606 by using the blockchain manager 614. Similarly, the node 600 may use the blockchain manager 614 in conjunction with the NFTs 628 and/or the smart contracts 616 stored in the memory 604 to provide the functionality disclosed herein. The memory 604 may further include chain data 624 including, for example, a state database of the blockchain for storing states of smart contracts deployed thereon.

In other embodiments, the smart contracts 616 operate independent of the blockchain manager 614 or other applications. In some embodiments, the node 600 does not have a blockchain manager 614, NFTs 628, or smart contracts 616 stored at the node. In some embodiments, the node 600 may have additional or fewer components than described.

### Exemplary Methods for Implementing a Medical Data NFT

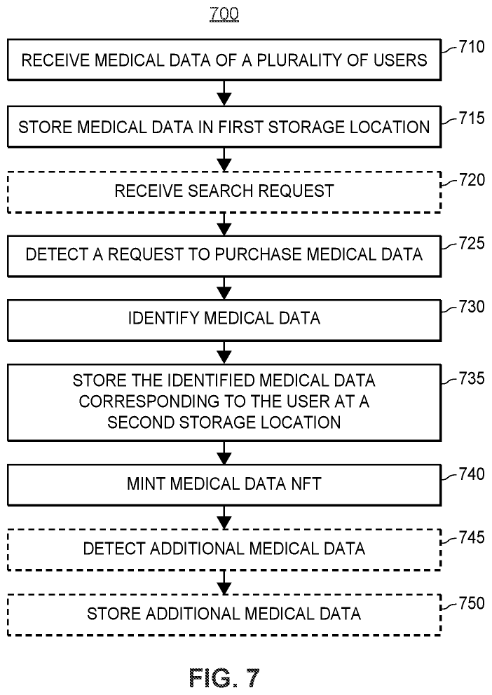

FIG. 7 shows an exemplary computer-implemented method or implementation 700 of implementing a medical data NFT. The exemplary method or implementation 700 may be performed by the one or more processors 120 of the medical data NFT server 102. Additionally or alternatively, any of the blocks of the exemplary method or implementation 700 may be performed by one or more nodes of the distributed ledger 130, including nodes that enforce smart contracts of the distributed ledger 130.

The method may begin at block 710 when the one or more processors 120 receive medical data of a plurality of users, such as a plurality of users including user 170. The medical data may be any kind of medical data. Examples of the medical data include heart rate data, blood oxygen level data, disease history, treatment history, mental health history, user characteristic data (e.g., the user's height, weight, gender, socio-economic status, etc.), etc.

The medical data may be received from any suitable source. For example, the medical data may be received from the user device 175 (e.g., a smart watch that gathers heart rate data, etc.), the electronic medical record (EMR) system 196, the hospital database 197, etc. In some embodiments, the received medical data is tagged with one or more labels that indicate, for example, a type and/or subtype of the medical data. Examples of the labels include heart rate data, blood oxygen level data, disease history, treatment history, user characteristic data (height, weight, gender, socio-economic status, etc.), and so on.

Additionally, the one or more processors 120 may obtain indications of prices at which the user is willing to sell medical data and/or particular types thereof. For example, a user 170 may offer to sell blood pressure data for one price, and medical history data at another price. In some embodiments, the user 170 may deploy a data owner smart contract that indicates the prices for the various types of medical data (such as via the user interface 200 of FIG. 2). For example, the data owner smart contracts may be configured to include a data structure (e.g., an XML, data structure) that indicates the medical data available for purchase, the price of the medical data, any restrictions the use of the medical data, and so on.

At block 715, the one or more processors 120 store the received medical data of the plurality of users in a first storage location. In some embodiments, the first storage location is in the medical data database 118. Additionally or alternatively, the first storage location may be in a secure third party database separate from the medical data NFT server 102, such as the third party database 195.

At optional block 720, the purchaser 160 may conduct a search for medical data made available via the medical data NFT server 102. In one example, the purchaser device 161 may be a node of distributed ledger 130. In this example, the purchaser device 161 may perform a search of the local copy of the distributed ledger 130 to identify medical data made available via data owner smart contracts. For example, the purchaser device 161 may perform the search of data owner smart contracts in response to detecting an interaction with the search button 312 of the user interface 300 described with respect to FIG. 3A.

In this example, the purchaser device 161 may search any field included in the data owner smart contract data structure. For example, the search may include fields that indicate a type of medical data (e.g., heart rate data, etc.), a collection period (e.g., data that is not more than 3 years old, etc.), an update frequency (e.g., daily, hourly, static, etc.), a price (e.g., in a traditional currency (U.S. dollars, Euros, etc.), a cryptocurrency (e.g., Bitcoin, USD Coin etc.), etc.), and so on. Additionally, the search may indicate any search terms entered via a search bar. In response, the purchaser device 161 may identify one or more data owner smart contracts that match the search criteria.

In another example, the purchaser 160 instead searches for users that have medical data maintained with the medical data NFT server 102, but not made available via a data owner smart contract. For example, the purchaser 160 may want to transmit an offer to a data owner that has a particular type of medical data maintained with the medical data NFT server 102. For example, the purchaser device 161 may send the search request in response to detecting an interaction with the search button 380 of the user interface 350 described with respect to FIG. 3B. In these embodiments, the purchaser device 161 may transmit a request to the medical data NFT server 102 to conduct a search of the user profiles maintained thereat. In response, the one or more processors 120 may provide information on the matching users to the purchaser device 161.

At block 725, the one or more processors 120 may detect a request, from a requestor (e.g., the purchaser 160, etc.), to purchase a data feed for one or more indicated types of medical data of a user 170 of the plurality of users. In some embodiments, the purchase request is a transaction on the distributed ledger 130 formatted to cause a data owner smart contract to self-execute to initiate a purchase medical data purchase process. More particularly, the request may identify one or more feeds of medical data and their corresponding usage restrictions made available for purchase via the data owner smart contract. In these embodiments, the purchase may occur in a decentralized manner.

In other embodiments, the purchaser 160 may send an offer to user(s) to purchase their medical data. For example, the purchaser 160 may cause the one or more processors 120 to generate a push notification via an application executing on the user device 175. The application may then enable the user 170 to accept, reject, and/or propose a counter-offer for their medical data.

In some embodiments, if the user 170 accepts an offer to purchase their medical data, the application sends a notification to the medical data NFT server 102 to process the purchase and/or set up the purchased data feed. In other embodiments, the application writes a transaction to the distributed ledger 130 indicating the agreed upon terms. In these embodiments, the one or more processors 120 may detect the transaction as the trigger to process the purchase and/or set up the purchased data feed.

At block 730, the one or more processors 120 begins processing the detect purchase by identifying, in the first storage location, the medical data for the user 170 that corresponds to the purchased medical data. In some embodiments, the identification is done according to labels placed on the medical data. For example, if the purchase request is for a user's heart rate data, the one or more processors 120 may identify a general data storage location for medical data associated with the user and then identify the specific data storage location for the user's heart rate data.

At block 735, the one or more processors 120 store the identified medical data corresponding to the user 170 at a second storage location, such as a storage location in the medical data database 118, the third party database 195, and/or a file sharing network 190. Additionally or alternatively, the second storage location may be on the medical data NFT itself.

At block 740, the one or more processors mint the medical data NFT that includes a reference to the second storage location (e.g., a storage location in the medical data database 118, the third party database 195, and/or a file sharing network 190). The minting of the medical data NFT may include adding the medical data NFT to a digital wallet associated with the requestor (e.g., the purchaser 160). Additionally or alternatively, the medical data may be stored on the medical data NFT during the minting process.

Furthermore, in some embodiments, the medical data NFT corresponds to multiple types of medical data of the user 170. In some such examples, multiple data feeds from the same user are bundled together. For example, data feeds including: (i) heart rate data from a smart watch (e.g., the user device 175), (ii) an EMR from the EMR system 196, and/or, (iii) blood oxygen level data from the hospital database 197 may all be bundled together such that the minted medical data NFT corresponds to any or all of the data feeds.

In some examples, the medical data NFT may include a hash of the medical data corresponding to the user, which may be used to prove that the NFT references the purchased medical data and that the version of the medical data stored at the second location has not been inappropriately modified. For example, the medical data engine 124 may calculate a hash of the data it stores in the second storage location and update the hash included in the NFT. The purchaser device 161 may calculate hash of the purchased medical data to ensure that hashes match, thereby authenticating the purchased medical data.

Furthermore, in some examples, the one or more processors 120 may configure a smart contract associated with the medical data NFT to include a set of rights associated with the medical data stored at the second storage location. More particularly, the one or more processors 120 may configure the smart contract to include the purchased set of rights may indicated by the purchase request detected at block 725. For example, the set of rights may include a temporal restriction on the access to the medical data, a usage restriction on the medical data (e.g., data may be used for medical research purposes, but not marketing purposes, etc.), and/or a transferability restriction on the medical data NFT (e.g., that the medical data may not be sold downstream, or may only be sold downstream a predetermined number of times, etc.).

Furthermore, the same or different smart contract may be configured to enforce payment terms for access to the indicated types of medical data. For example, the smart contract may be configured to self-execute to transfer currency to a digital wallet (e.g., of the user 170) when the user's 170 medical data is sold to the purchaser 160. To this end, the smart contract may be configured to self-execute to record transaction(s) on the blockchain that transfer currency. In one such example, the smart contract may be configured to self-execute to record a transaction that transfers currency from the purchaser 160 to the user 170. That is, in some embodiments, the enforcement of the payment terms may include transferring payment from a digital wallet associated with an owner of the medical data NFT to a digital wallet associated with the user 170.

At optional block 745, the one or more processors 120 detect that additional medical data of the type made available via the medical data NFT was added to the medical data database 118. For example, heart rate data collected from a smart watch of the user 170 may be regularly uploaded to the medical data database 118. In response, the one or more processors 120 may store the additional data at the second storage location indicated by the medical data NFT (block 750).

It should be understood that not all blocks and/or events of the example of FIG. 7 are required to be performed, or to be performed in the order specified. Furthermore, the example of FIG. 7 may include additional, less, or alternate functionality, including that discussed elsewhere herein.

### Additional Exemplary Embodiments for Implementing a Medical Data NFT

In one aspect, a computer system for implementing a medical data non-fungible token (NFT), may be provided. The computer system may include one or more local or remote processors, sensors, transceivers, servers, memory units, mobile devices, augmented reality glasses or headsets, extended or virtual reality headsets, smart glasses or watches, wearables, and/or other electronic or electrical components. In one instance, the computer system may include: a medical data database configured to store medical data, and/or one or more processors; and/or one or more memories coupled to the one or more processors. The one or more memories including computer executable instructions stored therein that, when executed by the one or more processors, may cause the one or more processors to: (1) receive medical data of a plurality of users; (2) store the received medical data of the plurality of users in a first storage location, wherein the first storage location is in the medical data database; (3) detect a request from a requestor to purchase a data feed for one or more indicated types of medical data of a user of the plurality of users; (4) identify, in the medical data database, the medical data for the user that matches the one or more indicated types of medical data; (5) store the identified medical data corresponding to the user at a second storage location; and/or (6) mint a medical data NFT that indicates the second storage location, wherein minting the medical data NFT adds the medical data NFT to a digital wallet associated with the requestor. The computer system may include additional, less, or alternate functionality, including that discussed elsewhere herein.

In some embodiments, the second storage location is in the medical data database, a third party database separate from the medical data database, or a file sharing network.

In some embodiments, the one or more memories having stored thereon computer executable instructions that, when executed by the one or more processors, cause the one or more processors to: detect that additional medical data of the one or more indicated types of medical data was added to the medical data database; and/or store the additional medical data at the second storage location.

In some embodiments, to receive the medical data, the one or more memories having stored thereon computer executable instructions that, when executed by the one or more processors, further cause the one or more processors to: receive the medical data from: (i) smart devices corresponding to the plurality of users, and/or (ii) an electronic medical records (EMR) system.

In some embodiments, the medical data corresponding to the user includes medical data labeled as heart rate data, blood oxygen level data, disease history data, and/or treatment history data.

In some embodiments, the medical data corresponding to the user includes user characteristic data including data of the user's: height, weight, gender, and/or socio-economic status.

In some embodiments, user records in the medical data database are configured to associate medical data types with a price at which the user is willing to sell a data feed of the corresponding medical data type.

In some embodiments, the one or more memories having stored thereon computer executable instructions that, when executed by the one or more processors, further cause the one or more processors to: receive, from the requestor, a price query for one or more medical data types associated with the user; and/or provide, to the requestor, a quote to the requestor based upon the prices at which the user is willing to sell the data feeds for the one or more medical data types included in the price query.

In some embodiments, the distributed ledger includes a data owner smart contract configured to indicate a price at which a data owner is willing to sell medical data.

In some embodiments, to mint the medical data NFT, the one or more memories having stored thereon computer executable instructions that, when executed by the one or more processors, further cause the one or more processors to: configure a smart contract associated with the medical data NFT to include a set of rights associated with the medical data stored at the second storage location.

In some embodiments, the set of rights include a temporal restriction on the access to the medical data, a usage restriction on the medical data, or a transferability restriction on the medical data NFT.

In some embodiments, to mint the medical data NFT, the one or more memories having stored thereon computer executable instructions that, when executed by the one or more processors, further cause the one or more processors to: configure a smart contract associated with the medical data NFT to self-execute to enforce payment terms for access to the indicated types of medical data.

In some embodiments, enforcing the payment terms for access to the indicated types of medical data includes transferring payment from a digital wallet associated with an owner of the medical data NFT to a digital wallet associated with the user.

In another aspect, a computer-implemented method for implementing a medical data non-fungible token (NFT) may be provided. The method may be implemented via one or more local or remote processors, sensors, transceivers, servers, memory units, mobile devices, augmented reality glasses or headsets, extended or virtual reality headsets, smart glasses or watches, wearables, and/or other electronic or electrical components. The computer-implemented method may include: (1) receiving, via one or more processors, medical data of a plurality of users; (2) storing, via the one or more processors, the received medical data of the plurality of users in a first storage location, wherein the first storage location is in a medical data database; (3) detecting, via the one or more processors, a request from a requestor to purchase a data feed for one or more indicated types of medical data of a user of the plurality of users; (4) identifying, via the one or more processors, in the medical data database, the medical data for the user that matches the one or more indicated types of medical data; (5) storing, via the one or more processors, the identified medical data corresponding to the user at a second storage location; and/or (6) minting, via the one or more processors, a medical data NFT that indicates the second storage location, wherein minting the medical data NFT adds the medical data NFT to a digital wallet associated with the requestor. The method may include additional, fewer, or alternate actions, including those discussed elsewhere herein.

In some embodiments, the second storage location is in the medical data database, a third party database separate from the medical data database, or a file sharing network.

In some embodiments, the computer-implemented method may further include: detecting, via the one or more processors, that additional medical data of the one or more indicated types of medical data was added to the medical data database; and/or storing, via the one or more processors, the additional medical data at the second storage location.

In some embodiments, receiving the medical data includes: receiving, via the one or more processors, the medical data from: (i) smart devices corresponding to the plurality of users, and/or (ii) an electronic medical records (EMR) system.

In some embodiments, the medical data corresponding to the user includes medical data labeled as heart rate data, blood oxygen level data, disease history data, and/or treatment history data.

In some embodiments, the medical data corresponding to the user includes user characteristic data including data of the user's: height, weight, gender, and/or socio-economic status.

In some embodiments, user records in the medical data database are configured to associate medical data types with a price at which the user is willing to sell a data feed of the corresponding medical data type.

### Other Matters

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A computer system configured to implement a medical data non-fungible token (NFT), the computer system comprising:
a medical data database configured to store medical data; and
one or more processors; and
one or more memories;
the one or more memories having stored thereon computer-executable instructions that, when executed by the one or more processors, cause the one or more processors to:
receive medical data of a plurality of users;
store the received medical data of the plurality of users in a first storage location, wherein the first storage location is in the medical data database;
detect a request from a requestor to purchase a data feed for one or more indicated types of medical data of a user of the plurality of users;
identify, in the medical data database, the medical data for the user that matches the one or more indicated types of medical data;
store the identified medical data corresponding to the user at a second storage location; and
mint a medical data NFT that indicates the second storage location, wherein minting the medical data NFT adds the medical data NFT to a digital wallet associated with the requestor.

2. The computer system of claim 1, wherein the second storage location is in the medical data database, a third party database separate from the medical data database, or a file sharing network.

3. The computer system of claim 1, wherein the one or more memories having stored thereon computer executable instructions that, when executed by the one or more processors, cause the one or more processors to:
detect that additional medical data of the one or more indicated types of medical data was added to the medical data database; and
store the additional medical data at the second storage location.

4. The computer system of claim 1, wherein to receive the medical data, the one or more memories having stored thereon computer executable instructions that, when executed by the one or more processors, further cause the one or more processors to:
receive the medical data from: (i) smart devices corresponding to the plurality of users, and/or (ii) an electronic medical records (EMR) system.

5. The computer system of claim 1, wherein the medical data corresponding to the user includes medical data labeled as heart rate data, blood oxygen level data, disease history data, and/or treatment history data.

6. The computer system of claim 1, wherein the medical data corresponding to the user includes user characteristic data including data of the user's: height, weight, gender, and/or socio-economic status.

7. The computer system of claim 1, wherein user records in the medical data database are configured to associate medical data types with a price at which the user is willing to sell a data feed of the corresponding medical data type.

8. The computer system of claim 7, wherein the one or more memories having stored thereon computer executable instructions that, when executed by the one or more processors, further cause the one or more processors to:
receive, from the requestor, a price query for one or more medical data types associated with the user; and
provide, to the requestor, a quote to the requestor based upon the prices at which the user is willing to sell the data feeds for the one or more medical data types included in the price query.

9. The computer system of claim 1, wherein:
the distributed ledger includes a data owner smart contract configured to indicate a price at which a data owner is willing to sell medical data.

10. The computer system of claim 1, wherein to mint the medical data NFT, the one or more memories having stored thereon computer executable instructions that, when executed by the one or more processors, further cause the one or more processors to:
configure a smart contract associated with the medical data NFT to include a set of rights associated with the medical data stored at the second storage location.

11. The computer system of claim 10, wherein the set of rights include a temporal restriction on the access to the medical data, a usage restriction on the medical data, or a transferability restriction on the medical data NFT.

12. The computer system of claim 1, wherein to mint the medical data NFT, the one or more memories having stored thereon computer executable instructions that, when executed by the one or more processors, further cause the one or more processors to:
configure a smart contract associated with the medical data NFT to self-execute to enforce payment terms for access to the indicated types of medical data.

13. The computer system of claim 12, wherein enforcing the payment terms for access to the indicated types of medical data includes transferring payment from a digital wallet associated with an owner of the medical data NFT to a digital wallet associated with the user.

14. A computer-implemented method for implementing a medical data non-fungible token (NFT), comprising:
receiving, via one or more processors, medical data of a plurality of users;
storing, via the one or more processors, the received medical data of the plurality of users in a first storage location, wherein the first storage location is in a medical data database;
detecting, via the one or more processors, a request from a requestor to purchase a data feed for one or more indicated types of medical data of a user of the plurality of users;
identifying, via the one or more processors, in the medical data database, the medical data for the user that matches the one or more indicated types of medical data;
storing, via the one or more processors, the identified medical data corresponding to the user at a second storage location; and
minting, via the one or more processors, a medical data NFT that indicates the second storage location, wherein minting the medical data NFT adds the medical data NFT to a digital wallet associated with the requestor.

15. The computer-implemented method of claim 14, wherein the second storage location is in the medical data database, a third party database separate from the medical data database, or a file sharing network.

16. The computer-implemented method of claim 14, further comprising:
detecting, via the one or more processors, that additional medical data of the one or more indicated types of medical data was added to the medical data database; and
storing, via the one or more processors, the additional medical data at the second storage location.

17. The computer-implemented method of claim 14, wherein receiving the medical data comprises:
receiving, via the one or more processors, the medical data from: (i) smart devices corresponding to the plurality of users, and/or (ii) an electronic medical records (EMR) system.

18. The computer-implemented method of claim 14, wherein the medical data corresponding to the user includes medical data labeled as heart rate data, blood oxygen level data, disease history data, and/or treatment history data.

19. The computer-implemented method of claim 14, wherein the medical data corresponding to the user includes user characteristic data including data of the user's: height, weight, gender, and/or socio-economic status.

20. The computer-implemented method of claim 14, wherein user records in the medical data database are configured to associate medical data types with a price at which the user is willing to sell a data feed of the corresponding medical data type.
